(19) United States
(12) Patent Application Publication
(10) Pub. No.: US 2006/0211902 A1
(43) Pub. Date: Sep. 21, 2006

(54) Palladium modified metal-promoted catalyst

(76) Inventors: Xin Xiao, Houston, TX (US); James Butler, League City, TX (US); Charles Comeaux, Beaumont, TX (US); Kevin Kelly, Friendswood, TX (US)

Correspondence Address: FINA TECHNOLOGY INC, PO BOX 674412, HOUSTON, TX, 77267-4412, US

(21) Appl. No.: 11/082,445

(22) Filed: Mar. 16, 2005

(51) Int. Cl. C07C 5/22; C07C 5/52

(52) U.S. Cl. 585/475; 585/266

(57) ABSTRACT

Processes for the conversion of an aromatic substrate over a mordenite catalyst modified by the inclusion of nickel and a second Group 10 metal selected from palladium, platinum and mixtures thereof. A feedstock containing at least one aromatic compound is brought into contact with the catalyst under conditions effective for the conversion of the aromatic compound to at least one derivative product. The resulting reaction product is then removed from the modified mordenite catalyst. The conversion reaction includes a toluene disproportionation reaction in which the feedstock contains toluene and the product recovered from the catalyst contains benzene and xylene. The conversion reaction can also involve a transalkylation reaction in which a mixture of benzene and xylene is brought into contact with the nickel and palladium-modified mordenite to produce a product which contains a monoalkyl benzene. The conversion can also involve the methylation of benzene to produce toluene or the methylation of toluene to produce xylene. The modified mordenite catalyst may contain nickel in an amount greater than the amount of platinum or palladium in the catalyst.

### BACKGROUND OF THE INVENTION

A number of aromatic conversion reactions involving the conversion of an aromatic compound to produce one or more reaction products are known. The disproportionation of toluene involves a well known transalkylation reaction in which toluene is converted to benzene and xylene in accordance with the following reaction, which is mildly exothermic:

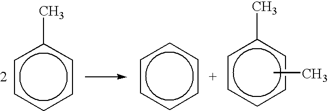

Another transalkylation reaction involves the reaction of an aromatic compound with a polyalkylated aromatic compound to produce a monoalkylated aromatic compound. One transalkylation reaction involves the transalkylation of a dialkylbenzene, such as xylene and benzene to produce a monoalkylated benzene such as toluene.

Conversion reactions involve the production of an alkyl benzene, e.g., the reaction of benzene and a methylating agent to produce toluene and the hydrogenation of an aromatic compound to the corresponding cycloalkane, such as the hydrogenation of benzene to produce cyclohexane.

Mordenite is one of a number of catalysts which can be employed in the transalkylation of alkylaromatic compounds. Mordenite is a crystalline aluminosilicate zeolite having a network of silicon and aluminum atoms interlinked in its crystalline structure through oxygen atoms. For a general description of mordenite catalysts, reference is made to Kirk-Othmer Encyclopedia of Chemical Technology, 3^{rd} Edition, 1981, under the heading “Molecular Sieves,” vol. 15, pages 638-643. Mordenite, as found in nature or as synthesized, typically has a relatively low silica to alumina mole ratio of about 10 or less. Such conventionally structured mordenite catalysts are commonly employed in the disproportionation of toluene. However, aluminum deficient mordenite catalysts having substantially lower alumina contents can also be employed in the disproportionation of toluene.

The aluminum deficient mordenite catalysts have a silica/alumina ratio greater than 10 and may sometimes range up to about 100. Such low alumina mordenites may be prepared by direct synthesis as disclosed, for example, in U.S. Pat. No. 3,436,174 to Sand or by acid extraction of a more conventionally prepared mordenite as disclosed in U.S. Pat. No. 3,480,539 to Voorhies et al. U.S. Pat. No. 3,780,122 to Pollitzer discloses the transalkylation of toluene using a mordenite zeolite having a silica/alumina ratio greater than 10 which is obtained by acid extraction of a mordenite zeolite having a silica/alumina ratio of less than 10.

The disproportionation of toluene feedstocks may be carried out at temperatures ranging from about 200° C. to about 600° C. or above and at pressures ranging from atmospheric to perhaps 100 atmospheres or above. However, the catalyst itself may impose constraints on the reaction temperatures in terms of catalyst activity and aging characteristics. In general, the prior art indicates that while relatively high temperatures can be employed for the high aluminum mordenites (low silica to alumina ratios), somewhat lower temperatures should be employed for the low alumina mordenites. Thus, where mordenite catalysts having high silica/alumina ratios have been employed in the transalkylation of alkylaromatics, it has been the practice to operate toward the lower end of the temperature range. It is also a common practice in this case to promote the catalyst with a catalytically active metallic content, as disclosed, for example, in U.S. Pat. No. 3,476,821 to Brandenburg. Metal promoters are said to substantially increase activity and catalyst life and may be incorporated by treatment of the mordenite with metal sulfides such as nickel sulfide.

Hydrogen may be supplied along with the toluene to the reaction zone. While the disproportionation reaction (1) is net of hydrogen, the use of a hydrogen co-feed is generally considered to prolong the useful life of the catalyst, as disclosed, for example, in the above-identified patent to Brandenburg. The amount of hydrogen supplied, which can be measured in terms of the hydrogen/toluene mole ratio or in terms of a standard liter of hydrogen per liter of feedstock, is generally shown in the prior art to increase as temperature increases. Normally, the hydrocarbon feedstock supplied to the toluene disproportionation reaction zone is of extremely high purity. Typically, feedstocks having a toluene content of 90-99 wt. % are supplied to the reaction zone. Usually, it is considered desirable to maintain the toluene content in excess of 99 wt. % (less than 1% impurities) in order to avoid unacceptably rapid catalyst deactivation.

### SUMMARY OF THE INVENTION

In accordance with the present invention, there are provided novel processes for the conversion of an aromatic substrate over a mordenite catalyst which has been modified by the inclusion of nickel and a second Group 10 metal selected from the group consisting of palladium, platinum and mixtures thereof. In carrying out the invention, a feedstock containing at least one aromatic compound is brought into contact with the modified mordenite catalyst under conditions effective for the conversion of the aromatic compound to at least one derivative product. The resulting reaction product is then removed from the modified mordenite catalyst.

In one embodiment of the invention, the conversion reaction involves a toluene disproportionation reaction in which the feedstock contains toluene and then product recovered from the catalyst contains benzene and xylene. In another embodiment of the invention, the conversion reaction involves a transalkylation reaction in which a feedstock comprising a mixture of benzene and dialkyl benzene is brought into contact with the nickel and palladium-modified mordenite to produce a product which contains a monoalkyl benzene. A specific application of this transalkylation process is the transalkylation of benzene and xylene to produce toluene.

In the foregoing aromatic conversion reactions, the nickel and palladium/platinum-modified mordenite catalyst preferably will contain nickel in an amount which is greater than the amount of platinum or palladium in the catalyst. Specifically, the second Group 10 metal will be present in an amount which is less than one-half the amount of nickel in the catalyst. More specifically, the mordenite catalyst will contain nickel in a concentration within the range of 0.1-2 wt. % and palladium in an amount within the range of 0.001-0.1 wt. %.

A further application of the present invention involves the hydrogenation of an aromatic compound to produce the corresponding cycloalkane. A specific application of this embodiment of the invention involves the hydrogenation of toluene to produce methylcyclohexane. In this embodiment of the invention, the catalyst will contain a minor amount of nickel and a predominant amount of the other Group 10 metal. Specifically, the catalyst may take the form of a mordenite catalyst having a palladium content of 0.001-0.5 wt. % and a lower amount of nickel in a concentration within the range of 10-500 ppm.

### BRIEF DESCRIPTION OF THE DRAWINGS

FIG. 1 is a graphical presentation of yields and toluene conversion as a function of time for a palladium-modified mordenite catalyst.

FIG. 2 is a graphical presentation of yields and toluene conversion as a function of time for a palladium/nickel-modified mordenite catalyst.

### DETAILED DESCRIPTION OF THE INVENTION

The present invention involves the use of Group 10 modified mordenite catalysts in aromatic conversion reactions involving the alkylation, transalkylation and hydrogenation of one or more aromatic reactants. The reactions are carried out over a modified mordenite catalyst which incorporates nickel and at least one other Group 10 metal selected from a group consisting of palladium and platinum and mixtures thereof. The preferred Group 10 metal, which is employed in addition to nickel, is palladium. However, the invention may be carried out with the inclusion of platinum in a nickel-modified mordenite catalyst or the inclusion of a mixture of palladium and platinum in a nickel-modified mordenite catalyst.

A preferred embodiment of the present invention provides a process that can be employed to disproportionate toluene to produce benzene and xylene. The toluene disproportionation catalyst employed in the present invention is provided by the inclusion of palladium in a nickel-modified mordenite, such as disclosed in U.S. Pat. No. 6,706,937, the entire disclosure of which is incorporated herein by reference. The nickel mordenite catalyst typically has a nickel content within the range of 0.1 -2 wt. % and also includes an other Group 10 metal, specifically palladium or platinum, and may also include lanthanide series metals, specifically lanthanum and cerium. Thus, the nickel-modified mordenite catalyst contains, in addition to the nickel, another Group 10 metal and may contain at least one lanthanide series metal. Suitable disproportionation procedures and mordenite catalysts which can be employed in the present invention are also disclosed in U.S. Pat. Nos. 4,956,511, 5,387,732, and 5,475,180, the entire disclosures of which are incorporated herein by reference.

The mordenite catalyst employed in the present invention may be natural mordenites of relatively low silica/alumina ratios of about 10 or less. Such catalysts are disclosed in the aforementioned Kirk-Othmer Encyclopedia of Chemical Technology, 3^{rd} Edition, 1991, vol. 15, pp. 638-643, the entire disclosure of which is incorporated herein by reference. Alternatively, alumina-deficient mordenite catalysts typically having a silica/alumina mole ratio within the range of about 10-100 can be used in carrying out the present invention.

A suitable nickel-modified mordenite catalyst which can be employed in the present invention comprises a catalyst in the form of extruded pills, which comprise about 70-80% of the zeolite mordenite and about 20-30% of a binder, normally in the form of alumina, which is composited with the zeolitic mordenite. In manufacturing such catalysts, the mordenite and binder are composited together and then extruded and dried, followed by calcination to further dry out the mordenite. The catalyst is then impregnated with nickel by any suitable technique such as disclosed in the aforementioned U.S. Pat. No. 4,956,511 to Butler et al.

The palladium and/or platinum may be incorporated into the nickel-modified catalyst concurrently with or subsequent to impregnation of the catalyst with nickel. For the aromatic conversion reactions involving transalkylation, the nickel typically will be employed in an amount within the range of 0.1-2 wt. % of the mordenite catalyst and the palladium or platinum incorporated into the catalyst in somewhat lower concentrations than the nickel. Where palladium is employed as the co-promoter, it will usually be employed in an amount within the range of 0.005-0.03 wt. % based upon the mordenite catalyst. The platinum will usually be employed in somewhat lower amounts, typically within the range of 0.001-0.03 wt. %.

The toluene disproportionation reaction may be carried out under conditions to provide toluene conversion within the range of about 40-55% as disclosed in U.S. Pat. No. 6,706,937 to Xiao, et al., the entire disclosure of which is incorporated herein by reference. Conversion can be at the rate of about 46-47% when employing a nickel-modified mordenite catalyst having a nickel content of about 1 wt. %. In general, the process parameters disclosed the aforementioned U.S. Pat. No. 6,706,937 can be employed in the present invention.

In transalkylation reactions involving the transalkylation of benzene and xylene to produce toluene, the reactor may be operated within the range of about 100-500° C. The reactor conditions may provide for a gas phase transalkylation, but normally the reactor pressure will be at a temperature of about 200° C. or above to provide that the aromatic components, benzene and diethyl benzene, are in the liquid phase.

Where the reaction carried out in accordance with the present invention involves the hydrogenation of an aromatic component such as benzene to produce the corresponding cycloalkane, cyclohexane, the reaction conditions typically will be in the temperature range of 50-450° C. and pressure within the range of 0.1-5 MPa. The hydrogen co-feed may be employed to provide a mole ratio of hydrogen to the aromatic substrate within the range of about 1-3. Here, the preferred relationship between the relative amounts of nickel and palladium (or platinum, if platinum is employed) is reversed from that described previously. Thus, in the hydrogenation process, the modified mordenite catalyst will contain palladium in amount within the range of 0.05-0.5 wt. % and nickel in a lower concentration, within the range of 0.005-0.05 wt. %.

### EXAMPLES

Experiments were conducted to examine the catalytic properties of palladium-modified mordenite catalysts; characterize the physical and catalytic properties of a nickel-promoted mordenite; and investigate the effect of palladium modification of a nickel-promoted mordenite. The test system used was for the catalysis of toluene disproportionation.

### Example 1

A first experiment was performed to compare the catalytic activity and selectivity of palladium-modified mordenite and nickel-promoted mordenite catalysts. The base mordenite catalyst comprised between about 50 and about 90% mordenite and the balance alumina binder. The mordenite catalyst was impregnated with about 0.1 wt. % palladium to produce a palladium-promoted mordenite catalyst. For comparative purposes, a nickel-promoted mordenite, containing about 1 wt. % nickel, was also studied.

The metal impregnation was achieved by wetness incipit methods, similar to that described in U.S. Pat. No. 3,476,821 to Brandenburg et al., the entire disclosure of which is incorporated herein by reference. Palladium impregnation involved preparing a stock solution containing palladium nitrate dissolved in water. This stock solution was added to a calcined extrudate comprised of about 80 wt. % mordenite, and about 20 wt. % alumina as binder to provide a metal containing mordenite. After allowing the metal impregnated extrudate to dry (about 110° C., for about 16 hours), the extrudate was calcined (about 500° C., for about 3 hours).

The palladium mordenite and nickel mordenite catalysts were separately introduced into the reactor zone of a conventional fixed bed reactor for subsequent testing over several days time-on-stream. In the presence of hydrogen gas, a substantially pure toluene feedstock, i. e., greater than about 99% toluene was supplied to a reaction zone containing each of the above metal-promoted mordenite catalysts under temperature and inlet pressure conditions within the range of about 250 to about 400° C. and about 1 MPa (400 psig) to about 4.1 MPa. The pressure was about 4.1 MPa at startup and subsequently reduced to about 2.7 MPa after five days continuous time-on-stream. Hydrogen was supplied to the reaction zone in an amount to provide a hydrogen:toluene molar ratio (H₂:HC) of about 1:1. The feedstock liquid hourly space velocity (LHSV) was about 2 hr⁻¹.

For a reactor temperature and pressure maintained at between about 360 and 365° C., and about 2.7 MPa, respectively, the reaction over each catalyst resulted in about 47% conversion of the toluene feed into a product mixture, as indicated by an observed approximately 53% of toluene remaining in the reactor effluent. The product mixture was analyzed for xylenes, benzene, heavy aromatics, i.e., product having 9 carbon atoms or more, C₁ to C₅ gases (i.e., methane to pentane), and nonaromatics, using conventional methods. The palladium mordenite catalyst had greater selectivity towards the production of xylene as compared to the nickel mordenite catalyst.

Using the palladium mordenite catalyst, the product mixture (normalized to 100 wt. %), with the reactor maintained at steady state process conditions for at least about 5 days, comprised: about 48 wt. % xylenes; about 37 wt. % benzene; about 12 wt. % heavy aromatics; about 1 wt. % C₁ to C₅ gases; and about 2 wt. % nonaromatics. In comparison, a reactor bed comprising nickel mordenite as the catalyst, under similar process conditions, produced a product mixture comprising: about 46 wt. % xylenes, about 37 wt. % benzene, about 11.5 wt. % heavy aromatics, about 4 wt. % C₁ to C₅ gases, and about 1.5 wt. % nonaromatics. Thus, the palladium mordenite catalyst has about 2 wt. % greater selectivity to xylenes and about 3 wt. % less selectivity to C₁ to C₅ gases than the nickel mordenite catalyst.

Further experiments were performed to characterize the selectivity of the palladium mordenite catalyst for producing increased proportions of xylene during toluene disproportionation. A palladium mordenite catalyst containing about 0.1 wt. % of palladium was used. The reactor zone conditions were the same as described about with the following exceptions. Temperature and reactor inlet pressure were in the range of about 400 to about 500° C. and about 1 MPa, respectively. Hydrogen was supplied to the reaction zone in an amount sufficient to provide a hydrogen:toluene molar ratio of about 3:1 and the LHSV was about 7 hr⁻¹. The reaction was allowed to proceed for a time-on-stream of about 9 days during which the temperature was increased by about 15° C. per day.

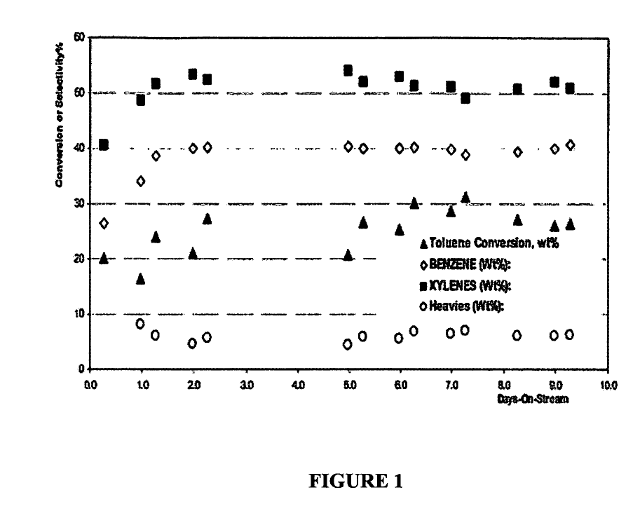

The time course of changes in the reactor temperature, percent conversion of toluene and the proportions of compounds in the product mixture are presented in FIG. 1 in which concentration in wt. % is plotted on the ordinate vs. time-on-stream in days on the abscissa. The conversion of toluene, ranging from about 16 to about 32%, was lower than the 47% conversion observed in the previously discussed experiment, due to the lower reactor pressure and higher LHSV. Similar to that discussed above, however, after about 2 days time-on-stream, the product mixture contained higher proportions of xylene (e.g., at least about 50%), as compared to that previously observed for the nickel mordenite catalyst. The reaction mixture also contained lower proportions of nonaromatics (e.g., less than about 2 wt. %) and C₉₊heavy aromatics (e.g., less than about 7 wt. %), as compared to that expected for the nickel mordenite catalyst. Average percentages from about 1.3 to about 9.3 days on-stream were: 39.9 wt. % benzene; 51.8 wt. % xylenes; 5.9 wt. % heavy aromatics; 0.2 wt. % ethylbenzene; and 2.2 wt. % nonaromatics.

Another preparation of the palladium mordenite catalyst was examined using the reactor conditions summarized in Table 1. Temperature and reactor inlet pressure were in the range of about 400 to about 450° C. and about 1 MPa to about 2.1 MPa. Hydrogen was supplied to the reaction zone in an amount sufficient to provide a hydrogen:toluene molar ratio of about 2.3:1 to about 3.3:1 and the LHSV was about 6.5 to about 9 hr⁻¹. As shown in Table 1, the conversion of toluene increased from about 14.6 to about 19% when the reactor bed temperature was increased from about 397 to about 445° C. And, the conversion increased to about 26% when the reactor pressure was increased to about 2.1 MPa (about 308 psig).

TABLE 1

| TOS | T | P | LHSV | | Conv. | Selectivity, wt. % | | | | |
|---|---|---|---|---|---|---|---|---|---|---|
| (d) | ° C. | psig | hr⁻¹ | H₂:HC | wt. % | n-Ar | EB | B | X | H |
| 0.2 | 397 | 151 | 7.87 | 2.68 | 14.56 | 4.83 | 0.25 | 37.71 | 51.09 | 6.13 |
| 0.9 | 421 | 151 | 7.87 | 2.68 | 16.77 | 3.85 | 0.11 | 39.53 | 52.18 | 4.32 |
| 1.2 | 445 | 151 | 8.95 | 2.35 | 19.10 | 1.76 | 0.10 | 40.56 | 53.22 | 4.36 |
| 1.9 | 445 | 151 | 8.95 | 2.35 | 16.05 | 1.51 | 0.07 | 40.53 | 53.92 | 3.96 |
| 7.0 | 447 | 306 | 6.49 | 3.25 | 24.68 | 1.77 | 0.11 | 40.54 | 51.94 | 5.64 |
| 8.0 | 447 | 308 | 6.46 | 3.26 | 26.30 | 1.86 | 0.11 | 40.81 | 51.53 | 5.68 |

### Example 2

A second experiment was conducted to characterize the physical properties and catalytic selectivity of the nickel mordenite catalyst during toluene disproportionation. Nickel sites and acid sites for different lots of a commercial nickel mordenite catalyst were characterized by conventional TPR measurements. The nickel mordenite catalyst (containing about 1 wt. % nickel) was introduced into the same reactor as used in Example 1. The reaction was allowed to proceed under process conditions similar to that described in Example 1, with the following exceptions. Temperature and reactor inlet pressure were in the range of about 300 to about 400° C. and at least about 4.1 MPa, respectively. Hydrogen was supplied to the reaction zone in an amount sufficient to provide a hydrogen:toluene molar ratio of about 2:1 and the LHSV was about 3 hr⁻¹.

The TPR curves for three representative lots, A, B and C, of nickel mordenite, respectively, were examined. The TPR cures had a large peak having a maximum between about 650 and about 670° C., is assigned to nickel tightly bound to the support (designated as “tightly bound nickel”). A relatively smaller peak having a maximum between about 290 and about 310° C., was assigned to nickel loosely bound to the support (designated as “loosely bound nickel”). For comparison, the curve for a non-nickel promoted support (labeled “none”) had no peak at temperatures above about 150° C. Based on calibration of the peaks, it is estimated that loosely bound nickel comprised about 1 to about 3% of the total nickel in the catalyst.

The toluene disproportionation catalyzed reaction typically reached the target conversion of about 47% toluene into the produced mixture at a reactor temperature of about 354° C. A typical product mixture comprised: about 47 wt. % xylenes; about 40 wt. % benzene; about 10 wt. % heavy aromatics; and about 0.6 wt. % ethylbenzene.

During the course of these experiments, it was discovered that a relationship exists between the toluene disproportionation catalyzed reaction and the extent of loosely bound nickel present in the catalysis, as characterized by the TPR results. For example, it was discovered that the time production of on-specification product was faster for nickel mordenite catalysts having relatively lesser amounts of loosely bound nickel. For example, lots A and B, having relatively prominent TPR peaks at about 300° C., took about eight days to reach on-specification levels of benzene. In contrast, lot C having minimal TPR peaks at about 300° C. took only about 2-3 days to reach on-specification yields of benzene. Moreover, it was discovered that lots having relatively prominent TPR peaks at about 300° C., also had relatively large amounts of undesirable nonaromatics in the product mixture during the early stages after starting the reaction (i.e., during about the first 3 to 7 days).

### Example 3

A third experiment was conducted to investigate the catalytic effects of palladium modification of a nickel-promoted catalyst and nickel modification of a palladium mordenite catalyst. The same nickel mordenite catalyst as described in Example 2 and containing about 1 wt. % nickel, was used. A solution containing a palladium salt was added to a nickel-containing mordenite extrudate such that the final palladium concentration was about 116 ppm per weight in the catalyst. The extrudate was then dried and calcined similar to Example 1 palladium-modified nickel-promoted mordenite catalyst.

The palladium modified nickel mordenite catalyst was then loaded into a reactor, and a stream of toluene was fed into the reactor over a period of from about day 1 to about day 17, and from about day 34 to about day 55. The toluene disproportionation process conditions, similar to that described above, were sufficient to provide about 46% conversion of toluene.

As illustrated in Table 2, the palladium modified nickel mordenite catalyst had toluene disproportionation activity with increased proportions of xylene production and decreased proportions of heavy aromatics, as compared to that typically observed for nickel mordenite. Average values for the product mixture from the palladium modified nickel mordenite catalyst comprised: about 40 wt. % benzene; about 48 wt. % xylenes; about 0.9 wt. % ethylbenzenes; about 10.6 wt. % heavy aromatics; and about 0.7 wt. % nonaromatics.

TABLE 2

Weight Percent

| | Toluene | |
|---|---|---|
| Component | Feed | Effluent |
| Nonaromatics | 0.02 | 0.30 |
| Benzene | 0.18 | 18.38 |
| Toluene | 99.48 | 53.90 |
| Ethylbenzene | 0.01 | 0.39 |
| p-Xylene | 0.02 | 5.23 |
| m-Xylene | 0.01 | 11.82 |
| o-Xylene | 0.03 | 5.08 |
| Cumene | 0.00 | 0.00 |
| Propylbenzene | 0.01 | 0.02 |
| Ethyltoluene | 0.04 | 0.66 |
| Trimethylbenzene | 0.08 | 2.82 |
| Diethylbenzene | 0.01 | 0.07 |
| Other C₁₀ | 0.09 | 0.05 |
| C₁₁₊ Heavy aromatics | 0.03 | 1.26 |

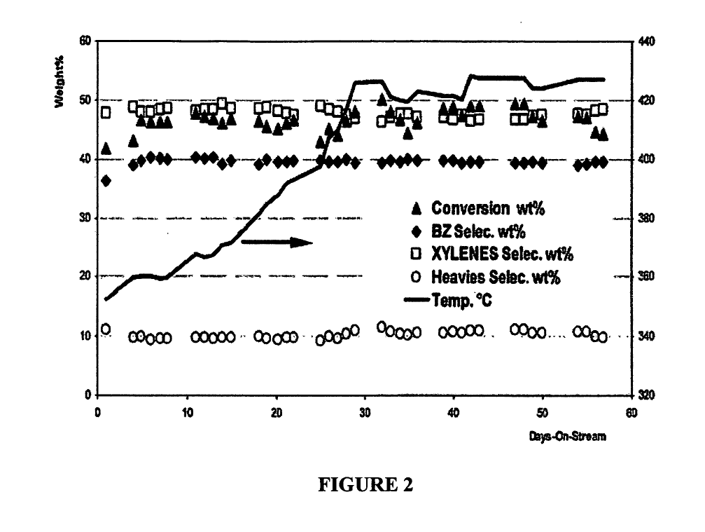

Yet another preparation of the palladium modified nickel mordenite catalyst was examined for 57 days of time-on-stream using a toluene feed and various process conditions. The palladium modified nickel promoted mordenite catalyst comprised about 76 ppm palladium and about 1 wt. % nickel on a mordenite having a SiO₂:Al₂O₃ molar ratio of about 90:1. Temperature and reactor inlet pressure were in the range of about 330 to about 500° C. and about 2 MPa to about 4.2 MPa, respectively. Hydrogen was supplied to the reaction zone in an amount sufficient to provide a hydrogen:toluene molar ratio (H₂:HC) of about 0.9:1 to about 3:1. The LSHV was about 2.8 to about 3.5 hr⁻¹. Specific process conditions for time-on-stream are presented in Table 3 and temperature and pressure adjustments are illustrated in FIG. 2 in which concentration in wt. % and temperature in degrees Centigrade are plotted on the ordinate vs. time in days on the abscissa.

TABLE 3

| TOS | T | P | LHSV | | Conv. | Selectivity, wt. % | | | | |
|---|---|---|---|---|---|---|---|---|---|---|
| (d) | ° C. | psig | hr⁻¹ | H₂:HC | wt. % | n-Ar | EB | B | X | H |
| 0.9 | 369 | 601 | 3.0 | 1.0 | 54.7 | 28.6 | 2.5 | 21.7 | 31.6 | 15.7 |
| 1.9 | 338 | 601 | 2.9 | 1.1 | 43.1 | 37.2 | 1.4 | 20.6 | 28.5 | 12.2 |
| 4.9 | 342 | 602 | 2.8 | 1.1 | 38.3 | 26.1 | 1.4 | 25.6 | 34.5 | 12.4 |
| 6.0 | 356 | 600 | 2.8 | 1.1 | 41.7 | 19.3 | 1.6 | 28.4 | 38.1 | 12.5 |
| 6.9 | 365 | 602 | 2.8 | 1.1 | 41.4 | 14.6 | 1.6 | 30.3 | 40.8 | 12.6 |
| 7.9 | 368 | 296 | 3.4 | 0.9 | 25.3 | 4.2 | 0.4 | 38.0 | 49.9 | 7.5 |
| 8.9 | 411 | 301 | 3.4 | 0.9 | 34.8 | 1.4 | 0.3 | 39.8 | 50.7 | 7.8 |
| 12.9 | 435 | 301 | 3.4 | 0.9 | 27.5 | 0.9 | 0.2 | 39.5 | 52.9 | 6.5 |
| 13.9 | 435 | 300 | 3.5 | 0.9 | 25.3 | 1.4 | 0.2 | 39.6 | 52.7 | 6.1 |
| 14.9 | 470 | 300 | 3.3 | 0.9 | 31.1 | 1.2 | 0.3 | 39.6 | 51.6 | 7.2 |
| 15.9 | 470 | 402 | 3.2 | 0.9 | 36.3 | 1.4 | 0.5 | 39.4 | 50.3 | 8.4 |
| 20.0 | 470 | 400 | 3.1 | 2.9 | 42.2 | 2.1 | 0.7 | 38.6 | 49.0 | 9.6 |
| 21.0 | 475 | 403 | 3.2 | 2.8 | 43.5 | 2.1 | 0.7 | 38.5 | 48.7 | 10.0 |
| 22.0 | 480 | 399 | 3.2 | 2.8 | 45.0 | 2.3 | 0.8 | 39.0 | 47.8 | 10.2 |
| 22.9 | 480 | 400 | 3.2 | 2.9 | 44.3 | 2.3 | 0.8 | 38.8 | 48.0 | 10.1 |
| 26.2 | 486 | 402 | 3.2 | 2.8 | 44.5 | 2.5 | 0.8 | 39.0 | 47.6 | 10.1 |
| 27.2 | 485 | 400 | 3.2 | 2.8 | 43.8 | 2.3 | 0.8 | 38.3 | 48.3 | 10.2 |
| 28.1 | 491 | 399 | 3.2 | 2.8 | 45.6 | 2.3 | 0.9 | 37.5 | 48.4 | 10.9 |
| 29.2 | 491 | 401 | 3.2 | 2.9 | 45.1 | 2.3 | 0.9 | 37.3 | 48.6 | 10.9 |

As shown in Table 3, high proportions of nonaromatics were present in the product mixture immediately after startup. After 7 days, the reactor pressure was reduced from about 600 to about 300 psig. Within about 2 days of the reduction in pressure, nonaromatics and heavy aromatics decreased to below about 2% and about 10%, respectively. At day 15, with a reactor temperature of about 470° C. and pressure of 300 psig the conversion was about 31%. Increasing the pressure to about 400 psig resulted in an increase in conversion to about 45%.

Similar to the above-described preparation of the palladium modified nickel mordenite catalyst containing about 116 ppm palladium, the palladium modified nickel mordenite catalyst containing about 76 ppm palladium also had toluene disproportionation activity with higher proportions of xylene and lower proportions of heavy aromatics production (FIG. 2), as compared with the previously studied nickel mordenite catalyst (see Examples 1 and 2). Up to about 25 days time-on-stream, the average values for the product mixture from this preparation of palladium modified nickel mordenite catalyst comprised: about 40 wt. % benzene; about 48 wt. % xylenes; about 1 wt. % ethylbenzenes; about 9.5 wt. % heavy aromatics; and about 1-2 wt. % nonaromatics. The proportion of xylene in the product mixture remained above about 50% from day 8 to about day 16. And, from day 20 to 29, the proportion of xylenes remained between about 48 and about 49%. By 30 days time-on-stream, the improved selectivity towards xylene production as compared to nickel mordenite disappeared, and by 57 days time-on-stream, the proportion of xylene in the product mixture decreased to about 44-45 wt. % and corresponding increase in the proportion of heavy aromatics.

In contrast, a preparation comprising a nickel-modified palladium catalyst had poor selectivity towards the production of benzene and xylene under toluene disproportionation reaction conditions. The same palladium promoted mordenite catalyst as described in Example 1, containing about 0.1 wt. % palladium, was used. A solution containing a nickel salt was added to the palladium mordenite containing extrudate, such that the nickel concentration was about 110 ppm in the extrudate. The extrudate was then dried and calcined as described in Example 1.

The resulting nickel modified palladium promoted mordenite catalyst was then introduced into the reactor and a toluene feed introduced for about 9 days. The temperature and reactor inlet pressure were in the range of about 310 to about 360° C. and about 4.1 MPa, respectively. Hydrogen was supplied to the reaction zone in an amount sufficient to provide a hydrogen:toluene molar ratio of about 1:1 and the LHSV was about 3 hr⁻¹.

As illustrated in Table 4, the nickel-modified palladium mordenite had toluene disproportionation activity with decreased proportions of both benzene and xylene in the product mixture, and increased proportions of nonaromatics as compared to palladium-modified nickel mordenite, palladium-modified mordenite or nickel promoted mordenite catalysts examined in previous experiments. Average values for the product mixture from the nickel-modified palladium mordenite comprised: about 20.6 wt. % benzene; about 28.5 wt. % xylenes; about 1.2 wt. % ethylbenzenes; about 10.7 wt. % heavy aromatics; and about 39 wt. % nonaromatics. The nonaromatics were very rich in methylcyclohexane.

TABLE 4

| TOS | T | P | LHSV | | Conv. | Selectivity, wt. % | | | | |
|---|---|---|---|---|---|---|---|---|---|---|
| (d) | ° C. | psig | hr⁻¹ | H₂:HC | wt. % | n-Ar | EB | B | X | H |
| 0.9 | 342 | 601 | 2.9 | 1.0 | 53.6 | 43.5 | 1.6 | 15.9 | 26.0 | 13.0 |
| 1.9 | 310 | 601 | 2.8 | 1.1 | 34.4 | 53.6 | 0.5 | 14.6 | 21.5 | 9.7 |
| 3.0 | 328 | 600 | 3.1 | 1.0 | 44.3 | 47.9 | 0.9 | 18.0 | 23.8 | 9.3 |
| 3.9 | 330 | 603 | 3.1 | 1.0 | 45.5 | 43.8 | 1.0 | 20.3 | 25.5 | 9.4 |
| 6.9 | 331 | 601 | 3.1 | 1.0 | 33.9 | 36.4 | 0.9 | 22.5 | 30.7 | 9.5 |
| 7.9 | 348 | 600 | 3.1 | 1.0 | 41.8 | 28.1 | 1.5 | 24.5 | 34.0 | 11.9 |
| 8.9 | 360 | 602 | 3.1 | 1.0 | 43.6 | 19.8 | 1.7 | 28.4 | 37.8 | 12.3 |

Having described specific embodiments of the present invention, it will be understood that modifications thereof may be suggested to those skilled in the art, and it is intended to cover all such modifications as fall within the scope of the appended claims.

1. A process for the conversion of an aromatic substrate comprising:
(a) providing a mordenite catalyst which has been modified by the inclusion of nickel and a second Group 10 metal selected from the group consisting of palladium, platinum and mixtures thereof;
(b) supplying a feedstock containing at least one aromatic compound into contact with said modified mordenite catalyst under conditions effective for the conversion of said aromatic compound to at least one product derived from said aromatic compound; and
(c) removing a reaction product from said modified mordenite catalyst.

2. The process of claim 1 wherein one of said nickel and said second Group 10 metal is present in said mordenite catalyst in an amount which is less than the other of said nickel and said second Group 10 metal.

3. The process of claim 2 wherein said second Group 10 metal is present in said catalyst in a concentration which is less than one-half of the concentration of said nickel in said catalyst.

4. The process of claim 3 wherein said mordenite catalyst contains nickel in an amount within the range of 0.1-2 wt. %.

5. The process of claim 4 wherein said mordenite catalyst contains palladium in an amount within the range of 0.001-0.1 wt. %.

6. The process of claim 1 wherein said conversion reaction is a toluene disproportionation reaction in which said feedstock contains toluene and said product contains benzene and xylene.

7. The process of claim 6 wherein said mordenite catalyst contains nickel in an amount within the range of 0.1-2 wt. % and palladium in a concentration which is less than one-half the concentration of nickel in said mordenite catalyst.

8. The process of claim 1 wherein said conversion reaction is a transalkylation reaction in which said feedstock contains a mixture of benzene and a dialkyl benzene and said product contains a monoalkyl benzene.

9. The process of claim 8 wherein said mordenite catalyst contains nickel in an amount within the range of 0.1-2 wt. % and palladium in a concentration which is less than one-half the concentration of nickel in said mordenite catalyst.

10. The process of claim 8 wherein said feedstock comprises benzene and xylene and said product comprises toluene.

11. The process of claim 10 wherein said mordenite catalyst contains nickel in an amount within the range of 0.1-2 wt. % and palladium in a concentration which is less than one-half the concentration of nickel in said mordenite catalyst.

12. The process of claim 1 wherein said feedstock comprises an aromatic compound which is supplied into contact with said catalyst in the presence of hydrogen and said product comprises a cycloalkane.

13. The process of claim 12 wherein said feedstock comprises benzene and said product comprises methylcyclohexane and cyclohexane.

14. The process of claim 13 wherein said catalyst contains palladium which is present in said mordenite catalyst in an amount which is greater than the amount of nickel in said catalyst.

15. A method for the disproportionation of a toluene feedstock to provide a disproportionation product containing xylene and benzene comprising:
(a) providing a catalytic reaction zone containing at least one catalyst bed comprising a mordenite disproportionation catalyst modified by the inclusion of nickel and palladium into said catalyst;
(b) supplying a toluene-containing feedstock into said reaction zone and into contact with said nickel and palladium-modified mordenite catalyst under temperature and pressure conditions effective to carry out the disproportionation of toluene to produce a disproportionation product containing benzene and xylene in the presence of said nickel and palladium-modified disproportionation catalyst; and
(c) withdrawing said disproportionation product from said reaction zone.

16. The process of claim 15 wherein said palladium is present in said catalyst in a concentration which is less than one-half of the concentration of said nickel in said catalyst.

17. The process of claim 15 wherein said mordenite catalyst contains nickel in an amount within the range of 0. 1-2 wt. %.

18. The process of claim 17 wherein said mordenite catalyst contains palladium in an amount within the range of 0.001-0.1 wt. %.

19. The process of claim 17 wherein said mordenite catalyst contains palladium in an amount within the range of 0.005-0.03 wt. %.
